(19) United States
(12) Patent Application Publication
(10) Pub. No.: US 2008/0102138 A1
(43) Pub. Date: May 1, 2008

(54) Replacement of Vitamins, Minerals and Neurotransmitter Losses from Tobacco Smoking

(76) Inventor: Harlan Clayton Bieley, West Palm Beach, FL (US)

Correspondence Address: JOHN C. SMITH, ESQ., 2499 GLADES ROAD, SUITE 113, BOCA RATON, FL, 33431, US

(21) Appl. No.: 11/969,199

(22) Filed: Jan. 3, 2008

Related U.S. Application Data
(63) Continuation-in-part of application No. 11/554,364, filed on Oct. 30, 2006.
(60) Provisional application No. 60/767,546, filed on Jun. 21, 2006.
(60) Provisional application No. 60/951,328, filed on Jul. 23, 2007.

(51) Int. Cl. A61K 33/30; A61P 43/00

(52) U.S. Cl. 424/641

(57) ABSTRACT

A compound for the simultaneous treatment of nicotine addiction, the side effects of nicotine withdrawal, such as excessive appetite, and detrimental health effects caused by loss of vital body substances such as vitamins, minerals, amino acids, co-factors and neurotransmitters which result from tobacco smoking. The first component is a bivalent negative sulfur compound from a group that includes, but is not limited to, alkyl sulfides, colloidal sulfur, hydropersulfides, organic thio compounds or their salts. The second component is a replenishment formula which is used to correct losses of those substances caused by tobacco smoke. The first and second components can be used alone, or in combination.

### CROSS-REFERENCE TO RELATED APPLICATIONS

This application is a Continuation-In-Part application of, and claims the benefit of, the now expired provisional patent application entitled “Smoking Cessation Treatment With Appetite Suppression”, filed May 21, 2006, bearing U.S. Ser. No. 60/767,546 and naming Harlan Clayton Bieley, the named inventor herein, as sole inventor, the contents of which are specifically incorporated by reference herein in its entirety; and the currently pending non-provisional patent application entitled “Smoking Cessation Treatment With Appetite Suppression”, filed Oct. 30, 2006, bearing U.S. Ser. No. 11/554,364 and naming Harlan Clayton Bieley, the named inventor herein, as sole inventor, the contents of which is specifically incorporated by reference herein in its entirety; and the currently pending provisional patent application entitled “Replacement of Vitamin, Mineral, and Neurotransmitter Losses From Tobacco Smoking”, filed Jul. 23, 2007, bearing U.S. Ser. No. 60/951,328 and naming Harlan Clayton Bieley, the named inventor herein, as sole inventor, the contents of which is specifically incorporated by reference herein in its entirety.

### BACKGROUND OF THE INVENTION

1. Technical Field

The present invention relates to treatments for tobacco smokers. In particular, it relates to a method and treatment for placement of essential vitamins, minerals, and nutrients which the body loses as a consequence of tobacco smoking. In particular, it supplements vitamins, minerals, amino acids, co-factors, and neurotransmitters which are depleted from the body due to tobacco smoking. In addition, it reduces cravings for nicotine and food cravings which typically result from nicotine withdrawal.

2. Background

The health problems associated with tobacco smoking have caused numerous individuals to attempt to end their use of tobacco products. However, as is well-known, tobacco products can be highly addictive due to the addictive quality of nicotine which is present in tobacco products. Many individuals attempt to quit smoking by simply stopping their use of tobacco products. Unfortunately, due to the strong addictive nature of nicotine, the abrupt cessation of tobacco usage (i.e., quitting “cold turkey”) fails in most cases. Quite often, individuals require more than willpower alone to free themselves from nicotine addiction. It would be desirable to have a method of aiding an individual to cease smoking without having to rely on substantial levels of willpower.

In addition to the difficulty related to cessation of smoking, attempts to stop smoking often result in the creation of secondary problems related to smoking. In particular, a fairly common side effect that individuals experience when attempting to quit smoking is a substantial increase in appetite. The increase in appetite results in undesirable increases in body weight. In turn, the extra body weight can result in a variety of unwanted health problems, as well as an undesirable change in the individual's appearance. Further, the undesirable increase in body weight can have a detrimental effect on an individual's willpower when attempting to quit smoking, because the additional weight can be very frustrating to an individual. In fact, it may even contribute to an individual's decision to abandon their attempt to quit smoking. It would be desirable to have a method of increasing an individual's chances of success by avoiding unwanted weight gain during the difficult process of overcoming addiction to nicotine.

To assist individuals attempting to quit smoking and free themselves of addiction to nicotine, a variety of methods have been tried. One such method has been the use of nicotine supplements to reduce nicotine craving by the individual. Nicotine supplements can take several forms. For example, a nicotine chewing gum has been developed which permits individuals to satisfy the body's craving for nicotine without damaging their lungs by inhaling tobacco smoke. Likewise, nicotine patches which adhere to an individual's skin for transdermal absorption of nicotine have also been developed. The object of nicotine supplements is to satisfy an individual's craving for nicotine, without the highly negative health consequences of inhaling tobacco smoke. While nicotine supplements protect the individual from the significant harm that smoking causes by satisfying the need to obtain nicotine without inhaling tobacco smoke, they do not overcome the individual's addiction to nicotine. In addition, once the chewing gum is discarded, or the transdermal patch is no longer used, the craving for tobacco products often returns. As a result, nicotine supplements solve only some of problems associated with smoking. It would be desirable to have a method of weaning an individual away from smoking tobacco, while at the same time minimizing the numerous undesirable side effects of a withdrawal from smoking.

More recently, alternative treatments which do not use nicotine supplements have been developed to assist individuals in breaking the tobacco habit. Nicotine is a neural chemical which attaches to specific cell receptor sites in the brain. Therefore, preventing its ability to attach to these cell receptor sites is one method used to interfere with the addictive qualities of nicotine. One such alternative treatment involves the use of chemical compounds which bind to these cell receptor sites, and thereby prevent nicotine from binding to those cell receptor sites. As a result, the nicotine based cravings of the individual are reduced. A number of anti-smoking compounds have been found to be effective for this purpose. These compounds include alkyl sulfides, colloidal sulfur, hydropersulfides, organic thio compounds or their salts. The preferred thio compounds are thioglycerols, thioglycols or their salts.

One example of a commercially available product containing at least one of these compounds is Sulfonil™. Sulfonil is described in U.S. Pat. No. 4,596,706 as a method of controlling craving for tobacco or controlling tobacco withdrawal symptoms. The product uses bivalent negative sulfur compounds which attach to the individual's cell receptor sites, and thereby prevent nicotine from binding to those same cell receptor sites.

By removing or reducing the ability of the nicotine to bind with cell receptor sites, the smoker can gradually eliminate the addiction to nicotine. However, while products such as Sulfonil help an individual to eliminate the nicotine craving, it does nothing to address the side effects of nicotine withdrawal, namely excessive appetite and unwanted weight gain. It would be desirable to have a product which helps an individual overcome nicotine addiction, and also helps an individual avoid the unwanted side effects of nicotine withdrawal.

Another serious disadvantage associated with the use of tobacco, in addition to the addiction problems discussed above, is the depletion of vitamins, minerals, amino acids, co-factors and neurotransmitters which are a direct result of smoking tobacco products. Depending on an individual's particular body chemistry, and the number and types of vitamins, minerals, amino acids, co-factors and neurotransmitters which are lost due to cigarette smoking, a variety of physical and health problems may arise.

Tobacco smoke is inherently dangerous. However, cigarette smoke can be more so because of the additional factor of the burning paper wrapper that carries the tobacco itself. Cigarette smoke is extremely complex from a chemical point of view. In particular, there are approximately 4,000 chemicals which are present in cigarette smoke. At least 60 of these chemicals are known carcinogens. Further, cigarettes have also been shown to carry toxic and heavy metals which are dangerous to human health. In addition to the obvious dangers associated with carcinogens, cigarette smoke impacts the body's ability to function normally by interfering with important body resources, either directly or from toxic metals. The additional health impact caused by the diminution and depletion of important vitamins, minerals, amino acids, co-factors and neurotransmitters can lead to a wide range of health problems in the smoker. As stated, tobacco is not the only part of the cigarette which carries dangerous chemicals. In particular, the paper used by the cigarette manufacturers has been engineered to burn slowly. This can be accomplished, among other ways, by the addition of antimony to the paper. However, while this makes the paper burn slowly, it also places toxic materials such as antimony into the smoker's body.

In addition to the toxic material ingested when an individual smokes, there is another important side effect which impacts health in a significant way. In particular, there is a significant loss of vital substances, namely vitamins, minerals, amino acids, co-factors and neurotransmitters which are a direct result of smoking. Therefore, in addition to the obvious problems associated with smoking, such as cancer, there is a general loss of vital substances necessary for good health. When individuals attempt to quit smoking, they typically experience problems related to weight gain which impacts health. Likewise, if they do not quit smoking they will experience problems related to loss of vital substances required by the body for good health. It would be desirable to have a single product which would help an individual in both situations. Namely, a product which would help an individual to quit smoking by reducing cravings for nicotine and reducing excessive appetite which is associated with nicotine withdrawal, and simultaneously provide an individual with vital substances needed for good health which will help to replace those vital substances which are lost while smoking.

While the prior art has addressed some of the problems related to cessation of smoking, it has failed to address other problems, such as excessive weight gain, which can occur during the course of withdrawal from nicotine addiction. Likewise, the prior art has failed to provide a product to replenish vital substances lost by the body due to tobacco smoking. It would be desirable to have a product which is capable of addressing the basic problem of withdrawal from nicotine addiction, as well as subsequent withdrawal related side effects such as excessive weight gain which in turn leads to other health problems. Likewise, the prior art has failed to address problems, created by smoking tobacco, which lead to depletion of important body substances which are vital to an individual's health. Namely, vitamins, minerals, amino acids, co-factors and neurotransmitters.

### SUMMARY OF THE INVENTION

The present invention provides targeted amino acid therapy for placement of losses of neurotransmitters related to tobacco smoking. It provides a compound for the simultaneous treatment of nicotine addiction and undesirable side effects such as excessive appetite and weight gain that occur during the nicotine withdrawal process. The first component of the compound includes at least one bivalent negative sulfur, in an amount sufficient to control the craving or the withdrawal symptoms resulting from nicotine withdrawal. The bivalent negative sulfur is selected from a group that includes, but is not limited to, hydropersulfides, alkyl sulfides, colloidal sulfur, organic thio compounds or their pharmaceutically acceptable salts. The most effective thio compounds have proven to be thioglycerols, thioglycols or their pharmaceutically acceptable salts.

The second component of the compound relates to cravings, appetite suppression, and control of blood sugar. In the preferred embodiment, appetite suppression is accomplished using amino acids derived from tryptophan, such as 5-HTP. 5-HTP and/or related tryptophan derivatives. These are known in the art to suppress appetite and cravings for certain carbohydrates when ingested. The appetite suppression compounds are combined with the bivalent negative sulfur compound(s) to provide a single compound that produces the desirable effect of reducing nicotine craving while simultaneously suppressing increased appetite which is a result of nicotine withdrawal.

The third component of the compound includes a variety of nutritional supplements specifically selected to replenish vital body substances lost as a direct result of tobacco smoking. Some of the vital body substances depleted by tobacco smoking include: (a) vitamin C (ascorbic acid), (b) folic acid/folate, (c) vitamin B12, (d) serotonin, (e) magnesium, (f) vitamin E (e.g. alpha tocopherol, etc.), (g) biotin, (h) melatonin, (i) zinc, (j) selenium, (k) sulfhydryl groups (e.g. cysteine, glutathione), (l) n-acetyl cysteine (NAC) and (m) dopamine.

The cause of the depletion of vital body substances is due to the many toxic substances in tobacco smoke. Cigarette smoke includes heavy metals such as cadmium which causes a variety of health problems. In the case of cadmium, this can be a long-term problem because the body stores it in fat and may retain it for substantial amount of time. Cadmium causes the depletion of zinc. It causes the depletion of vitamin D which has direct and indirect effects on bone turnover. It is nephrotoxic and can interfere with vitamin D metabolism.

It is known that GSH (glutathione) is decreased in smoking and GSH is a tripeptide of glycine, glutamate (glutamic acid) and the all important, cysteine (L-cysteine is not well absorbed in the intestine). NAC is useful because it provides another way to increase intracellular glutathione via elevated intracellular cysteine.

Another health factor created by smoking is a reduction in the amount of vitamin C available to the body. Vitamin C has an effect on blood flow, and a reduction in blood flow caused by reduced amounts of vitamin C can directly impact the health in a number of ways. Tobacco smoke contains nickel which has the effect of reducing available levels of vitamin C in the body. In addition to nickel, tobacco smoke also contains cobalt. Nickel and cobalt has been shown to greatly deplete the level of intracellular ascorbate.

Tobacco smoking also depletes vitamin E in the body. A loss of vitamin E, and a loss of vitamin C contribute to a variety of health problems. In particular, the inflammatory process used by the body can be impacted, and in addition, plasma and urinary CEHCs can be decreased. CEHC (alpha CEHC=apha-carboxy-ethyl-hydroxychroman) is a metabolite of vitamin E.

Tobacco smoking also depletes sulfhydryl groups (e.g. cysteine, glutathione) which impacts adhesion and cellular morphology.

Tobacco smoking depletes brain dopamine. The inventive compound provides tyrosine and phenylalanine which are both precursors to dopamine production.

These compounds are combined with suitable binders to form pharmaceutical capsules that can be administered orally. Alternatively, these compounds can be administered via injection, via transdermal cream, gel or patch, sublingually, via gum or lozenge, or by any other suitable means. In some administration methods, such as gum or lozenge, sweetening agents, such as sugar alcohols, can be added to sweeten the taste without effecting blood sugar levels. Sugar alcohols, such as xylitol, can be used in combination with gums or lozenges to provide sweetness with a lower carbohydrate level than a comparative amount of sugar. As a result, it will not elevate blood sugar levels as much as sugar will. In addition, stevia and agave may also be used as sweeteners. Stevia and agave can both be useful in controlling blood sugar levels.

A therapeutic dosage level is approximately 120 milligrams, twice a day, of the bivalent negative sulfur, and multiple doses of the bivalent negative sulfur may be taken at the same time. Regarding 5-HTP, typical doses would run from a low dosage level of 50 milligrams to a high dosage level of 900 milligrams per day. It is important to note that the dosage level of each component of the compound can vary based on the size/weight of the individual in question, vary based on the tolerance level of that individual for components of the compound, or vary based on the intensity of the craving for nicotine or food. As a result, a small individual, or an individual sensitive to the compound, may achieve adequate results with small dosage levels of 5-HTP (e.g. less than 50 milligrams) per day. Likewise, a large individual, or an individual with a high tolerance level for the compound, may safely take more than what would typically be considered the maximum dosage of 5-HTP (e.g., greater than 900 milligrams) per day.

The treatment uses a compound which addresses both nicotine craving, and appetite control. The first component of the compound is designed to interfere with the ability of nicotine to attach to cell receptor sites, thereby reducing nicotine craving. The other component of the compound is directed to both nicotine craving and appetite control, thereby reducing excessive weight gain associated with withdrawal from nicotine addiction. The various components of the compound act together to assist an individual to quit smoking without the undesirable side effects of nicotine withdrawal, such as increased appetite and unwanted weight gain.

An advantage of tryptophan and tryptophan derivatives derives from the fact that when the body processes a tryptophan derivative such as 5-HTP, the tryptophan derivative is used by the body as a serotonin precursor to increase serotonin levels. It is the increased serotonin level that decreases appetite and craving for food which lead to excessive weight gain. An added benefit provided by serotonin is that it tends to decrease many types of craving, including craving for nicotine. As a result of its general affect on craving, it not only helps suppress appetite which leads to unwanted weight gain, but it also works synergistically with the sulfur based compounds to decrease craving for nicotine.

In addition, the invention provides compounds which supplement and assist the body to replenish and/or rebuild the body's vital substances, such as vitamins, minerals, amino acids, co-factors and neurotransmitters which are lost due to tobacco smoking. Vitamins, minerals, amino acids and neurotransmitters affected include vitamin C (ascorbic acid), folic acid, vitamin B6 (pyridoxal-5′-phosphate, pyridoxal), serotonin, magnesium, vitamin B12, Vitamin E in its various forms (such as alpha tocopherol, etc.) biotin, melatonin, dopamine, selenium (which may reduce the risk of lung cancer), coenzyme Q10 and zinc.

### DESCRIPTION OF THE PREFERRED EMBODIMENT

Prior to discussing detailed aspects of the invention, a general overview will be provided. The invention provides an improvement over prior art sulfur-based nicotine withdrawal products which block nicotine from binding with cell receptor sites. The prior art has focused its attention on the use of a variety of sulfur based compounds to treat nicotine addiction. However, it has not placed significant emphasis on the side effects associated with nicotine withdrawal. In particular, a frequent side effect associated with smoking cessation is a marked increase in appetite. The increase in appetite often results in an individual having the health benefits associated with cessation of smoking offset by the health hazards produced by excessive weight gain. These are increased insulin levels which can lead to insulin resistance, increased blood pressure, and unfavorable changes in lipid profiles.

The present invention overcomes these problems by providing a single therapeutic compound which simultaneously reduces nicotine craving to assist an individual to cease smoking, and simultaneously suppresses the individual's appetite to avoid unnecessary and undesirable side effects, such as weight gain, associated with the cessation of smoking.

The invention takes advantage of known sulfur based compounds which interfere with the binding of nicotine with cell receptor sites. These compounds, and their method of use, are discussed in detail in U.S. Pat. Nos. 4,416,869 and 4,596,706, which are incorporated by reference herein in their entirety. Applicant's invention improves over these prior art inventions by adding compounds which are specifically designed to reduce the increased appetite which is caused by nicotine withdrawal. Further, the compounds selected to reduce appetite also enhance the body's ability to suppress nicotine cravings. As a result, an individual seeking to cease smoking will not have the additional problems generated by the common side effect of nicotine withdrawal, namely, increased appetite and weight gain. Applicant's invention provides a single dose which is directed to both problems: overcoming nicotine addiction and reducing the side effect of increased appetite during nicotine withdrawal.

In addition to the ingredients that are directed to nicotine withdrawal and appetite suppression, the inventive compound also includes ingredients necessary to replenish and/or rebuild vital body substances, such as vitamins, minerals, amino acids, co-factors and neurotransmitters. Tyrosine and phenylalanine are dopamine precursors which are included in the compound to restore the level of dopamine for the purpose of improving brain function.

Having discussed the features and advantages of the invention in general, we turn now to a more detailed discussion of the figures.

As discussed above, the U.S. Pat. Nos. 4,416,869 and 4,596,706 teach a variety of sulfur based compounds which are useful in the treatment of nicotine addiction. In particular, these patents teach the use of bivalent negative sulfur compounds which are useful for reducing nicotine craving in individual's seeking to cease smoking tobacco products. However, these products do not effectively address side effects related to the cessation of smoking, most notably: unwanted weight gain due to increased appetite. The advantage of Applicant's invention is that it allows an individual to cease smoking without this undesirable side effect.

Applicant's invention provides a new compound which incorporates known compounds in the prior art related to suppression of nicotine addiction with a second compound which combined with those compounds to simultaneously suppress the unwanted increase in appetite which occurs during nicotine withdrawal. Applicant's invention uses derivatives of the amino acid tryptophan to reduce appetite so that an individual can cease smoking tobacco products without the added stress and health hazards related to excessive weight gain.

In the preferred embodiment, both the compounds related to nicotine addiction, and the amino acids used for appetite suppression are combined into a single dose for ingestion by the individual. Those skilled in the art will recognize that these compounds can be administered via a variety of methods, such as pills/capsule ingestion, liquid ingestion, intramuscular injection, intravenous injection, nasal spray, via inhaler, transdermal, suppository, or via powder mixed in a liquid. When used as a liquid, the compound can be administered in combination with any suitable solution, liquid carrier, liquid medium, etc. The only requirement is that liquid selected is suitable for use with the components of the compound. However, while a variety of administration methods can be used (e.g., injection, transdermal patches creams, gels, lozenges, gums, etc.), the preferred embodiment envisions the simple and convenient process of administering the compound via a pill or capsule.

In the preferred embodiment, the tryptophan derivative used by the Applicant is 5-hydroxytryptophan (“5-HTP”), which is a nutrient. It is derived from the amino acid L-Tryptophan. L-Tryptophan plays a vital role in our health. Tryptophan an essential amino acid for building proteins and enzymes, and serves as the precursor for serotonin, and the hydrogen carriers NADH and NADPH. 5-HTP functions as a precursor to serotonin (5-HT, 5-hydroxytryptamine). Serotonin, a neurotransmitter, plays an important role in regulation of mood, appetite, body temperature, and the secretion of various hormones. While serotonin does not readily cross the blood brain barrier, serotonin precursors such as 5-HTP can. Supplementation with this precursor increases levels of serotonin. In addition, 5-HTP is more efficient than L-tryptophan because it bypasses the rate-limiting step of serotonin synthesis (tryptophan hydroxylase).

Tryptophan has a variety of side effects when ingested by humans. It can be used as a mood-enhancer, and it can help individuals sleep. In fact, it has been widely used to treat insomnia and depression. It can increase pain tolerance, and it can also reduce appetite. An advantage of 5-HTP over some other appetite suppressants is that it has small molecule size. The 5-HTP accesses the brain from the bloodstream, and once in the brain, it can be converted into serotonin. It is the serotonin, created from the 5-HTP, acting on the different serotonin receptor sites, which ultimately acts as an appetite suppressant, and helps to reduce cravings for nicotine. A further advantage of 5-HTP over other potential appetite suppressants is that it is a naturally occurring compound which is produced in the body from tryptophan which is found in high-protein foods such as beef, chicken, fish, and/or dairy products.

In the preferred embodiment, the dosage level of 5-HTP ranges from 50 mg per day up to 900 mg per day. 700 to 900 mg of 5-HTP would normally be considered a high dose. Of course, those skilled in the art will recognize that there are many factors that influence the appropriate dosage level for an individual. For example, the size, weight and tolerance of individuals can vary widely. Therefore, an appropriate dosage for one individual may not be safe for another. A large individual may have the ability to use dosage levels well in excess of 900 mg. Some individuals may also have high tolerance levels for particular compounds which will result in the ability to use high dosage levels. Of course the opposite will be true for individuals who are small or particularly sensitive to a given compound. Those individuals may only need a dosage level less than 50 mg. In addition, other factors related to an individual may create higher or lower levels of appetite which would necessitate the change in dosage levels. As a result, unique factors related to each individual should be taken into account when determining the proper levels of each of the components of the compound.

In the preferred embodiment, the compound would be taken twice a day, to maintain stable levels of nicotine suppressant. In addition, it has been found that the appetite suppressant is more effective if taken before or after meals, because it is absorbed by the body more rapidly if taken on an empty stomach. As a result, an individual would preferably take the compound approximately an hour before meals, or approximately 2 hours after meals. Typically, an individual would take a dose twice a day. This would maintain relatively stable levels of nicotine suppressant throughout the day, and will also suppress hunger at the appropriate times.

Because 5-HTP is a precursor used by the body to produce serotonin which suppresses appetite, its use in combination with the aforementioned nicotine suppressant allows an individual to improve the chances of successfully overcoming a tobacco habit. This is because the individual will not have negative side effects such as increased weight gain, which may in fact frustrate the individual to the point where they resume smoking. Due to that, the individual is more likely to succeed when attempting to quit smoking. Of course, avoiding unnecessary weight gain provides many advantages for the overall health of the individual. In addition, because serotonin also inhibits cravings for nicotine, it will synergistically enhance the sulfur based compounds which are directed specifically at reducing nicotine cravings.

In addition to the use of 5-HTP to suppress appetite, other additives may be included in the compound to enhance weight control. For example, there are a number of nutritional supplements which enhance the body's ability to metabolize fat, such as vitamin B12, inositol, methionine, and choline. Additives such as this complement the suppressant of appetite by enhancing the body's ability to metabolize fat. Therefore, these additives would also help reduce the risk of weight gain during smoking cessation.

Inositol, in all of its forms, may be helpful in more than one way. Higher doses may help increase GABA and reduce anxiety. The prior art suggests that Inositol may increase neurotransmitters such as serotonin, and GABA. While medical literature suggests that individuals may safely ingest 1-18 grams per day, the preferred embodiment envisions a dosage level of approximately 0.4-2 grams/day.

Another effective additive is Dopamine, which is a neurotransmitter. L-Tyrosine 100-200 mg twice a day and L or DL-phenylalanine 300 mg a day will increase dopamine along with the B-Vitamins. The addition of the neurotransmitter precursors (tyrosine and phenylalanine) of dopamine may also help decrease cravings for carbohydrates. The L-phenylalanine form may work best. Other optional additives can also be usefully combined with the compound. For example, alpha-lipoic acid helps reduce cravings for sweets, which can contribute significantly to weight gain. Likewise, alpha-lipoic acid, chromium picolinate, the chromium product, Chromium Polynicotinate™, and biotin can be helpful in regulating blood sugar metabolism and may be included in the compound. Research suggests that chromium may help prevent glucose-induced elevation of systolic blood pressure and decreased measures of lipid peroxidation. High serum glucose levels which can also contribute to a destructive process known as glycosylation, also called nonenzymatic glycation, in which glucose molecules bind to proteins and interfere with their function. People with diabetes have a highly increased rate of protein glycosylation and this plays a major part in their increased risk of atherosclerosis and many other diseases. For many with diabetes, chromium enhances the ability of insulin to lower serum glucose levels. In addition to these nutritional supplements, some vitamins, mainly B6, may be used by the body in the process of converting 5-HTP to serotonin. Vitamin B6 also acts as a diuretic which further helps to control weight. Another possible additive is coenzyme Q10. Coenzyme Q10 is produced by the human body and is necessary for the basic functioning of cells. Tobacco smoke can deplete body stores of coenzyme Q10 (CoQ10), but levels of CoQ10 in the body can be increased by taking supplements.

In the preferred embodiment, the following daily doses are taken and used to supplement vital body substances lost because of tobacco smoking:
1. 50-200 mg 5-HTP (Serotonin precursor)
2. 50-250 mg Thioglycerol
3. 50-100 mg Choline Citrate
4. 50-100 mg Pantothenic Acid (as D-Calcium Pantothenate)
5. 400-2000 mg Inositol
6. 50-100 mg L-methionine
7. 200-500 mg Alpha Lipoic Acid
8. 30-500 mg Vitamin B-6 (as Pyridoxine HCl, or any of its other forms)
9. 2-10 gm Vitamin C
10. 800-2000 mcg Folic Acid
11. 800-10,000 mcg Vitamin B12 (as methylcobalamin)
12. 800-1600 mg magnesium (as magnesium citrate or magnesium glycinate)
13. 800-10,000 IU vitamin D3
14. 50-120 mg zinc (as zinc glycinate, picolinate, or citrate)
15. 100-1200 IU vitamin E (alpha-tocopherol and gamma tocopherol)
16. 200-400 mcg selenium (as selenomethionine)
17. 500-4000 mg tyrosine
18. 500-3000 mg phenylalanine (as L or DL-phenylalanine)
19. 300-600 mg coenzyme Q10
20. 0.1-6 mg melatonin
21. 30 mcg-2 mg biotin
22. 600-1200 mg N-acetyl cysteine

Those skilled in the art will recognize that while the aforesaid formula is preferred, changes can be made to the formula without losing its overall effectiveness. Regarding smoking cessation, two components of the compound are important. The first component is 5-HTP which is used to suppress appetite, and reduce cravings for tobacco. In the preferred embodiment, this is administered in an approximate dosage of 200 mg. The second compound is Thioglycerol, which is a sulfur based compound that is used to block nicotine receptor cells. By blocking the receptor cells, nicotine cravings are reduced. The Thioglycerol is typically administered in an approximate dosage of 120 mg. The combination of these two components provides a compound which reduces nicotine cravings while simultaneously reducing appetite. In addition, numerous ingredients listed above, which are intended to replenish vital body substances that are reduced through the use of tobacco can be used singly or in combination with one another. As a result, a single product can be used not only to assist an individual to quit smoking, to avoid the side effects according to tobacco such as weight gain, but simultaneously can be used by smokers to avoid damage done to the smoker's health by tobacco in the form of depletion of important compounds used by the body.

In addition to the two primary components, namely sulfur compounds used to block nicotine receptor cells, and the 5-HTP, which is used to increase serotonin levels of a variety of optional additional components can be used to enhance performance of the product. For example, weight suppression can be further enhanced through the use of Inositol, Vitamin B12 (as Methylcobalamin), L-methionine, and Choline Citrate. They can be used alone or in combination with one another to increase the body's ability to metabolize fat, and thereby helping the body to avoid weight gain. B vitamins, such as vitamin B-6 (as Pyridoxine HCl), Pantothenic Acid (as D-Calcium Pantothenate), and Folic Acid are precursors to, or co-factors for neurotransmitters, which will decrease cravings for certain carbohydrates. Alpha Lipoic Acid is helpful in stabilizing blood sugar levels. Magnesium Citrate may help to reduce anxiety.

Those skilled in the art will recognize that as is the case with any pharmaceutical or neutraceutical, appropriate dosages will vary based on several factors. A person's weight, age, physical condition, etc., will all influence what the proper dosage for a particular individual should be. As a result, while the foregoing dosages are envisioned as an appropriate starting point, changes can be made to suit particular individuals.

While the invention has been described with respect to a preferred embodiment thereof, it will be understood by those skilled in the art that various changes in detail may be made therein without departing from the spirit, scope, and teaching of the invention. For example, the compound used to suppress appetite can vary so long as it is suitable for its purpose, the amount of the appetite suppressant can vary based on an individual's physical requirements. The type of administration can vary from pill/capsule/powder form to liquid form, injection, transdermal patch, creams, gels, gums, lozenges, sublingual administration, etc. In addition, the dose and frequency of administration can vary based on the number of daily meals, severity of withdrawal systems, the weight and condition of the individual, etc.

In addition, the invention provides compounds which supplement and replace vital substances, such as vitamins, minerals, amino acids, co-factors and neurotransmitters which are lost due to tobacco smoking. Substances replaced or supplemented by this invention include vitamin C (ascorbic acid), folic acid, vitamin B6 (pyridoxal-5′-phosphate, pyridoxal), serotonin, magnesium, vitamin B12, vitamin E (Alpha tocopherol), Biotin, melatonin (a hormone and antioxidant), and zinc. Some substances, such as zinc and magnesium, can be absorbed by the body in several forms. For example, zinc can be absorbed as zinc glycinate, zinc picolinate, or those zinc citrate.

Cigarette smoke contains, among other things, lead, cadmium, nickel and antimony. One of the side effects of metals, such as lead, is that it decreases the dopamine levels in the body. That's why tyrosine and phenylalanine are included in the formula. They are dopamine precursors which the body uses to replace the dopamine that is lost due to smoking. In addition to lead, the cadmium which is found in cigarette smoke diminishes zinc levels in the body. Cadmium can be transferred into the fat cells and stored in the body for a substantial period of time. Further, cadmium can have direct and indirect effects on bone turnover, it can interfere with vitamin D metabolism, and it is a nephrotoxin.

It is also known that serotonin is excreted in urine at a higher rate in smokers than in non-smokers. As a result, by supplementing with 5-HTP, the body is better able to generate more serotonin to replace that lost due to tobacco use.

Vitamin C levels are also reduced due to tobacco smoking. For example, the nickel in tobacco smoke interferes with vitamin C which is used by the body to improve blood flow. Likewise, Vitamin E has been shown to disappear faster in blood plasma of smolders as opposed to non-smokers.

Those skilled in the art will recognize that while the aforesaid formula is preferred, changes can be made to the formula without losing its overall effectiveness. Two components of the compound are the most important. The first component is 5-HTP which is used to suppress appetite. In the preferred embodiment, this is administered in an approximate dosage of 200 mg. The second compound is Thioglycerol, which is a sulfur based compound that is used to block nicotine receptor cells. By blocking the receptor cells, nicotine cravings are reduced. The thioglycerol is typically administered in an approximate dosage of 120 mg. The combination of these two components provides a compound which reduces nicotine cravings while simultaneously reducing appetite.

The formula used above the combination of ingredients designed to address several problems created by tobacco smoking. By providing substantial amounts of these depleted substances, a smoker can replenish and restore the substances to more optimal levels for improved health. As a result, the user is able to prevent additional damage caused by tobacco due to the depletion of these substances. Those skilled in the art will recognize, as was the case above, that the exact dosage for every individual will vary based on factors such as body weight, age, smoking history, general health, etc. Likewise, those skilled in the art will also recognize that the formula used above can be combined with any of the above-described smoking cessation compounds such that while individuals are fighting their addiction to tobacco, they can simultaneously help their bodies maintain proper levels of vitamins, minerals, amino acids, co-factors and neurotransmitters.

Those skilled in the art will recognize that the invention includes compounds which may be used alone or in combination with smoking cessation embodiment, discussed above. As previously discussed, the heavy metals found in tobacco smoke, such as cadmium, can produce a number of undesirable side effects. For example, cadmium from tobacco smoke reduces brain dopamine, and possibly numerous other vital body substances which are necessary for health. As a result, it may be desirable to add additional components to the formula in order to compensate for these losses.

I claim:

1. A compound for treating vitamin, mineral, amino acid, co-factor and neurotransmitter related damage caused by tobacco smoke, comprising:
replacement vitamins, minerals, amino acids, co-factors and/or neurotransmitter precursors which replace or supplement the vitamins, minerals, co-factors and/or neurotransmitter precursors which are diminished in an individual's body as a side effect of smoking tobacco products;
whereby the replacement vitamins, minerals, co-factors and/or neurotransmitter precursors assist in the restoration of their normal levels within the individual's body.

2. A compound, as in claim 1, further comprising:
vitamin and vitamin-like supplements selected from the group of coenzyme Q10, vitamin B-6, vitamin C, folic acid, vitamin B12, vitamin D3, vitamin E, biotin, inositol, I-methionine, choline citrate;
mineral supplements selected from the group of magnesium, zinc, and selenium; and
neurotransmitter precursors, anti-oxidants, and hormones selected from the group of 5-HTP, thioglycerol, pantothenic acid, alpha lipoic acid, tyrosine, N-acetyl cysteine, melatonin and phenylalanine.

3. A compound, as in claim 2, wherein:
the vitamin supplement group contains an approximate daily dosage of 300-600 mg coenzyme Q10, and/or 30-500 mg vitamin B-6, and/or approximately 2-10 gm vitamin C, and/or approximately 800-2000 mcg folic acid, and/or approximately 800-10000 mcg vitamin B12, and/or approximately 800-10000 IU vitamin D3, and/or approximately 100-1200 IU vitamin E, and/or approximately 400-2000 mg inositol, and/or approximately 50-100 mg L-methionine, and/or approximately 50-100 mg choline citrate;
the mineral supplement group contains an approximate daily dosage of 800-1600 mg magnesium, and/or approximately 50-120 mg zinc, and/or approximately 200-400 mcg selenium; and
the neurotransmitter precursor, anti-oxidant, and hormone group contains an approximate daily dosage of 50-200 mg 5-HTP, and/or approximately 50-250 mg thioglycerol, and/or approximately 50-100 mg pantothenic acid, and/or approximately 200-500 mg alpha lipoic acid, and/or approximately 500-4000 mg tyrosine, 600-1200 mg N-acetyl cysteine, 0.1-6 mg melatonin and/or approximately 500-3000 mg phenylalanine.

4. A compound, as in claim 3, further comprising:
tryptophan and/or 5-HTP;
whereby the tryptophan and/or 5-HTP is used by the body as a serotonin precursor to decrease appetite and food craving by increasing serotonin levels.

5. A compound, as in claim 4, wherein:
the compound further comprises an approximate daily dosage of 50-200 mg of 5-HTP.

6. A compound, as in claim 5, further comprising:
thioglycerol;
whereby the thioglycerol is used by the body to reduce nicotine withdrawal symptoms.

7. A compound, as in claim 6, wherein:
the compound further comprises an approximate daily dosage of 50-250 mg of thioglycerol.

8. A compound, as in claim 3, further comprising:
thioglycerol;
whereby the thioglycerol is used by the body to reduce nicotine withdrawal symptoms.

9. A compound, as in claim 8, wherein:
the compound further comprises an approximate daily dosage of 50-250 mg of thioglycerol.

10. A compound, as in claim 2, further comprising:
tryptophan and/or 5-HTP;
whereby the tryptophan and/or 5-HTP is used by the body as a serotonin precursor to decrease appetite and food craving by increasing serotonin levels.

11. A compound, as in claim 10, wherein:
the compound further comprises an approximate daily dosage of 50-200 mg of 5-HTP.

12. A compound, as in claim 2, further comprising:
thioglycerol;
whereby the thioglycerol is used by the body to reduce nicotine withdrawal symptoms.

13. A compound, as in claim 12, wherein:
the compound further comprises an approximate daily dosage of 50-250 mg of thioglycerol.

14. A method minimizing the destructive effects of tobacco smoking, including the steps of:
replacing and/or supplementing vitamins, minerals, amino acids, co-factors, anti-oxidants and/or neurotransmitter precursors which are diminished in individual's body as a side effect of smoking tobacco products;
whereby the replacement vitamins, minerals, amino acids, co-factors, anti-oxidants, and neurotransmitter precursors assist in the restoration of their normal levels within the individual's body.

15. A method, as in claim 14, including the additional steps of:
replenishing vitamins, vitamin-like substances, minerals, amino acids, co-factors and neurotransmitter precursors by:
using one or more vitamin and vitamin-like supplements selected from the group of vitamin B-6, vitamin C, folic acid, vitamin B12, vitamin D3, vitamin E, biotin, inositol, and I-methionine, choline citrate;
using one or more mineral supplements selected from the group of magnesium, zinc, and selenium; and
using one or more neurotransmitter precursors, anti-oxidants, and/or hormones selected from the group of 5-HTP, melatonin, thioglycerol, pantothenic acid, alpha lipoic acid, tyrosine, and phenylalanine.

16. A compound, as in claim 15, wherein:
using daily dosages from the vitamin supplement group that contain approximately 30-500 mg vitamin B-6, and/or approximately 2-10 gm vitamin C, and/or approximately 800-2000 mcg folic acid, and/or approximately 800-10000 mcg vitamin B12, and/or approximately 800-10000 IU vitamin D3, and/or approximately 100-1200 IU vitamin E, and/or 30 mcg-2 mg biotin, and/or approximately 400-2000 mg inositol, and/or approximately 50-100 mg L-methionine, and/or approximately 50-100 mg choline citrate;
using daily dosages from the mineral supplement group that contain approximately 800-1600 mg magnesium, and/or approximately 50-120 mg zinc, and/or approximately 200-400 mcg selenium; and
using daily dosages from the neurotransmitter precursor, anti-oxidant, and hormone group that contain approximately 50-200 mg 5-HTP, and/or approximately 50-250 mg thioglycerol, and/or approximately 50-100 mg pantothenic acid, and/or approximately 200-500 mg alpha lipoic acid, and/or approximately 500-4000 mg tyrosine, and/or approximately 500-3000 mg phenylalanine, 600-1200 mg N-acetyl cysteine, and/or approximately 0.1-6 mg melatonin.

17. A method, as in claim 16, including the additional step of:
controlling appetite and food craving by increasing serotonin levels with tryptophan and/or 5-HTP.

18. A method, as in claim 17, including the additional step of:
using daily dosages of approximately 50-200 mg of 5-HTP.

19. A compound, as in claim 16, including the additional step of:
using thioglycerol to reduce nicotine withdrawal symptoms.

20. A method, as in claim 19, including the additional step of:
using daily dosages of approximately 50-250 mg of thioglycerol.
